(19) United States
(12) Patent Application Publication
(10) Pub. No.: US 2021/0022699 A1
(43) Pub. Date: Jan. 28, 2021

(54) X-RAY FLUOROSCOPE

(71) Applicant: Shimadzu Corporation, Kyoto (JP)

(72) Inventor: Shinsuke KANAZAWA, Kyoto (JP)

(21) Appl. No.: 16/522,343

(22) Filed: Jul. 25, 2019

(51) Int. Cl. A61B 6/00; A61B 6/06; G21K 1/04

(52) U.S. Cl. CPC A61B 6/542; G21K 1/046; A61B 6/06

(57) ABSTRACT

When an irradiation range control member is reflected in a region of interest, a control unit of an X-ray fluoroscope is configured to perform at least one of a first control to update a region of interest to a new region of interest by excluding a portion where the irradiation range control member is reflected in the region of interest and a second control to assign a predetermined pixel value to a pixel of a portion where the irradiation range control member is reflected in the region of interest.

### CROSS-REFERENCE TO RELATED APPLICATIONS

The related application number JP2017-008032, entitled “X-ray fluoroscope”, filed on Jan. 20, 2017 (publication date: Jul. 26, 2018), and invented by Shinsuke Kanazawa, upon which this patent application is based, is hereby incorporated by reference.

### BACKGROUND OF THE INVENTION

### Field of the Invention

The present invention relates to an X-ray fluoroscope, and more particularly to an X-ray fluoroscope configured to control an X-ray irradiation unit based on a region of interest on an X-ray fluoroscopic image.

### Description of the Background Art

Conventionally, an X-ray fluoroscope configured to control an X-ray irradiation unit based on a region of interest on an X-ray fluoroscopic image is known. Such an X-ray fluoroscope is disclosed in, for example, Japanese Unexamined Patent Application Publication No. 2010-273834.

In the above-mentioned Japanese Unexamined Patent Application Publication No. 2010-273834, an X-ray image diagnostic apparatus (X-ray fluoroscope) configured to control an X-ray irradiation unit is disclosed. The X-ray image diagnostic apparatus is provided with an X-ray irradiation unit for emitting X-rays toward a subject, an X-ray image receiving unit for receiving X-rays, a top board for placing a subject thereon, and a control unit configured to generate an X-ray fluoroscopic image based on an X-ray received image result by the X-ray image receiving unit and control the X-ray irradiation unit so that an image level (for example, the mean value of the luminance value of each pixel) in the region of interest on the generated X-ray fluoroscopic image substantially matches a preset image level.

That is, the control unit of the X-ray image diagnostic apparatus described in Japanese Unexamined Patent Application Publication No. 2010-273834 controls the X-ray irradiation unit based on the region of interest on the X-ray fluoroscopic image. Specifically, in this X-ray image diagnostic apparatus, the control unit is configured to determine the excess or deficiency of the X-ray intensity based on the collected image levels in the region of interest and adjust the X-ray intensity.

Further, conventionally, an X-ray fluoroscope provided with an irradiation range control member capable of narrowing the irradiation range of X-rays is known. The irradiation range control member restricts the irradiation range of X-rays by shielding a part of X-rays irradiated from the X-ray irradiation unit toward the subject. Note that the irradiation range control member is made of a substance having a high X-ray shielding rate.

In the X-ray image diagnostic apparatus (X-ray fluoroscope) of the above-mentioned Japanese Unexamined Patent Application Publication No. 2010-273834, the control unit controls the intensity of X-rays emitted from the X-ray irradiation unit so that the image level (e.g., the mean value of the luminance value of each pixel) within the region of interest on the generated X-ray fluoroscopic image approximately matches a preset image level (for example, the mean value of the luminance value of each pixel).

Here, in the case of providing an irradiation range control member to the X-ray image diagnostic apparatus of the above-mentioned Japanese Unexamined Patent Application Publication No. 2010-273834, it is considered that the irradiation range control member is sometimes reflected in the region of interest, which generates a portion where almost no X-ray is detected in the region of interest. As described above, in cases where there exists a portion where the irradiation range control member is reflected in the region of interest, it is considered that the control unit controls to increase the intensity of X-rays irradiated by the X-ray irradiation unit in accordance with the decrease in the X-ray intensity detection as the entire region of interest due to the reflection of the irradiation range control member in the region of interest. As a result, the X-ray dose in the portion where the irradiation range control member is not reflected in the region of interest becomes excessive, so the X-ray fluoroscopic image becomes excessively bright. As a result, there is a problem that the visibility of the X-ray fluoroscopic image is deteriorated.

### SUMMARY OF THE INVENTION

The present invention has been made to solve the aforementioned problems, and one object of the present invention is to provide an X-ray fluoroscope capable of suppressing deterioration of visibility of an X-ray fluoroscopic image even when an irradiation range control member is reflected in a region of interest.

In order to attain the aforementioned object, an X-ray fluoroscope according to one aspect of the present invention includes:

an X-ray irradiation unit configured to irradiate X-rays to a subject;

an X-ray image receiving unit configured to receive X-rays transmitted through the subject;

an irradiation range control member configured to narrow an irradiation range of the X-rays irradiated from the X-ray irradiation unit; and

a control unit configured to control an opening degree of the irradiation range control member by acquiring an X-ray fluoroscopic image based on an X-ray image of the X-ray image receiving unit and control irradiation intensity of X-rays irradiated from the X-ray irradiation unit based on a region of interest on the X-ray fluoroscopic image,

wherein when the irradiation range control member is reflected in the region of interest, the control unit is configured to perform at least one of a first control to update the region of interest to a new region of interest by excluding a portion where the irradiation range control member is reflected in the region of interest and a second control to assign a predetermined pixel value to a pixel of the portion where the irradiation range control member is reflected in the region of interest.

In the X-ray fluoroscope according to one aspect of the present invention, as described above, in cases where the irradiation range control member is reflected in the region of interest set on the X-ray fluoroscopic image, the control unit is provided in which at least one of a first control to update the region of interest to a new region of interest by excluding a portion where the irradiation range control member is reflected in the region of interest and a second control to assign a predetermined pixel value to a pixel of a portion where the irradiation range control member is reflected in the region of interest is performed. With this, when performing the first control, since the portion where the irradiation range control member is reflected in the region of interest is excluded, the excess and deficiency of the X-ray irradiation intensity can be acquired appropriately based on only the portion where the irradiation range control member is not reflected in the region of interest.

Further, when performing the second control, since the pixel value when there is no reflection of the irradiation range control member is assigned to the portion where X-rays are not detected due to the reflection of the irradiation range control member, the excess and deficiency of X-ray irradiation intensity can be appropriately acquired based on the region of interest. As a result, it is possible to suppress that the intensity of X-rays irradiated to fluoroscopically inspect a subject excessively rises due to the reflection of the irradiation range control member in the region of interest. With this, it is possible to suppress deterioration of visibility of the X-ray fluoroscopic image even in cases where the irradiation range control member is reflected in a region of interest. Further, since the X-ray irradiation intensity can be suppressed from being excessively increased, the exposure dose of the operator of the X-ray fluoroscope and the subject can be reduced.

In the X-ray fluoroscope according to one aspect of the present invention, preferably, the control unit is configured to acquire a portion where the radiation range control member is reflected in the region of interest on the X-ray fluoroscopic image acquired from the X-ray image receiving unit based on the opening degree of the irradiation range control member.

By configuring as described above, from the opening degree of the irradiation range control member, it is possible to easily acquire the portion where the irradiation range control member is reflected in the region of interest. Further, the control unit can easily acquire the opening degree of the irradiation range control member since the opening degree of the irradiation range control member is controlled by the control unit.

In the X-ray fluoroscope according to one aspect of the present invention, preferably, the control unit is configured to adjust the irradiation intensity of the X-rays irradiated from the X-ray irradiation unit based on a mean value or a maximum value of a pixel value in the updated region of interest or a pixel value of the entire region of interest to which a predetermined pixel value is assigned.

By configuring as described above, when controlling based on the mean value of the pixel value in the updated region of interest or the pixel value of the entire region of interest to which the predetermined pixel value is assigned, the X-ray intensity can be adjusted based on the pixel value of the entire region of interest. Further, in the case of controlling based on the maximum value of the pixel value in the updated region of interest or the pixel value of the entire region of interest to which the predetermined pixel value is assigned, for example, when considering the luminance as the pixel value, the X-ray intensity can be adjusted based on the brightest pixel value in the region of interest.

In the X-ray fluoroscope according to one aspect of the present invention, preferably, when the irradiation range control member is opened when performing the first control, the control unit is configured to control to update the region of interest to a new region of interest by returning a portion which was excluded from the updated region of interest among a portion where the irradiation range control unit is no longer reflected in the region of interest again to a new region of interest up to an initially set region of interest as a maximum region.

By configuring as described above, even when the irradiation range control member which has been once closed is opened again during the fluoroscopic inspection of the subject, the excluded portion is returned to the region of interest. Therefore, the control unit can update (expand) the region of interest in accordance with the expansion of the irradiation range. Further, since the region of interest is updated (expanded) up to the initially set region of interest as a maximum region, it is possible to prevent the region of interest from been expanded excessively beyond the appropriate range.

In the X-ray fluoroscope according to one aspect of the present invention, preferably, when performing the second control, the control unit is configured to assign a maximum value of the pixel value of a portion where the irradiation range control member is not reflected in the region of interest to the portion where the irradiation range control member is reflected in the region of interest.

By configuring as described above, since the maximum value of the portion where the irradiation range control member is not reflected in the region of interest is assigned to the portion where the irradiation range control member is reflected in the region of interest, it is possible to easily assign a hypothetical pixel value of the entire region of interest when there is no reflection of the irradiation range control member to the portion where the irradiation range control member is reflected in the region of interest.

Specifically, it is considered that there exists no subject in most of the portion where the irradiation is shielded by the irradiation range control member. Therefore, the maximum value of the pixel value of the portion where the X-ray irradiation range control member is not reflected, which is assumed to be a portion detected by the image receiving unit without being transmitted through the subject is assigned to the portion where the irradiation range control member is reflected in the region of interest. With this, even when the irradiation range control member is reflected in the region of interest, it is possible to appropriately adjust the X-ray irradiation intensity based on the entire region of interest to which the pixel value is assigned.

In the X-ray fluoroscope according to one aspect of the present invention, preferably, the control unit is configured to be able to switch between the first control and the second control in accordance with an imaging region of the subject. By configuring as described above, since it is possible to switch between the first control and the second control according to the imaging region of the subject, a more desirable X-ray fluoroscopic image can be acquired according to the situation and purpose of the portion to be fluoroscopically inspected.

### BRIEF DESCRIPTION OF THE DRAWINGS

FIG. 1 is a view showing an overall configuration of an X-ray fluoroscope according to an embodiment of the present invention.

FIG. 2 is a schematic view for explaining an inside of an MLC (irradiation range control member) of the X-ray fluoroscope according to the embodiment of the present invention.

FIG. 3A is a view for explaining a preset ROI (initially set region of interest) of a first control of the X-ray fluoroscope according to the embodiment of the present invention.

FIG. 3B is a view for explaining a state in which an MLC (irradiation range control member) is reflected in the preset ROI (initially set region of interest) of the first control of the X-ray fluoroscope according to the embodiment of the present invention.

FIG. 3C is a view for explaining setting of a ROI (region of interest) of the first control of the X-ray fluoroscope according to the embodiment of the present invention.

FIG. 4A is a view for explaining the ROI (region of interest) of the first control of the X-ray fluoroscope according to the embodiment of the present invention.

FIG. 4B is a diagram showing the relationship between the ROI (region of interest) and the position where the MLC (irradiation range control member) is reflected.

FIG. 4C is a diagram for explaining resetting of an ROI (region of interest).

FIG. 5 is a flowchart for explaining first control processing of the X-ray fluoroscope according to an embodiment of the present invention.

FIG. 6A is a view for explaining a preset ROI (initially set region of interest) of a second control of the X-ray fluoroscope according to the embodiment of the present invention.

FIG. 6B is a view for explaining a state in which an MLC (irradiation range control member) is reflected in the preset ROI (initially set region of interest) of the second control of the X-ray fluoroscope according to the embodiment of the present invention.

FIG. 6C is a view for explaining setting of a ROI (region of interest) of the second control of the X-ray fluoroscope according to the embodiment of the present invention.

FIG. 7 is a flowchart for explaining second control processing of the X-ray fluoroscope according to the embodiment of the present invention.

### DESCRIPTION OF PREFERRED EMBODIMENTS

Hereinafter, some embodiments embodying the present invention will be described with reference to the attached drawings.

### EMBODIMENT

### (Configuration of X-Ray Fluoroscope)

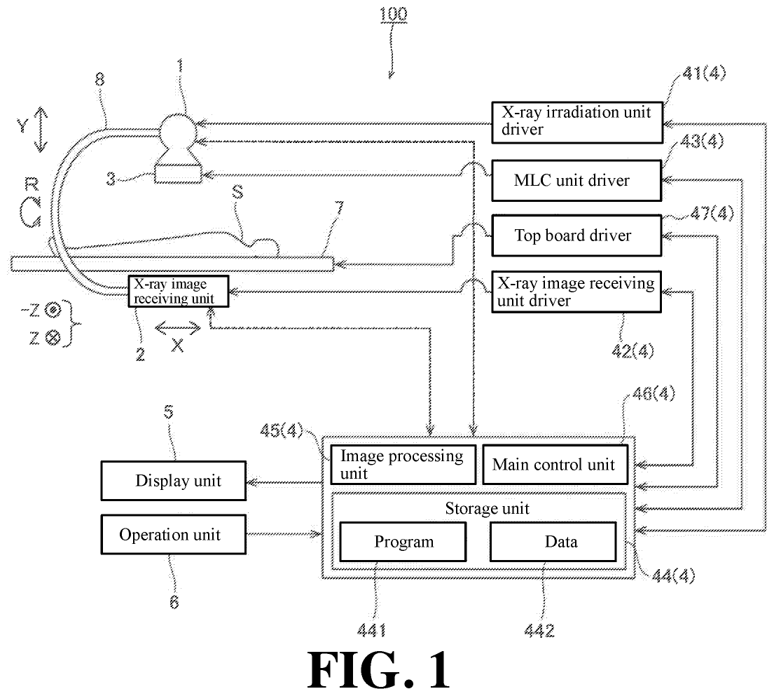

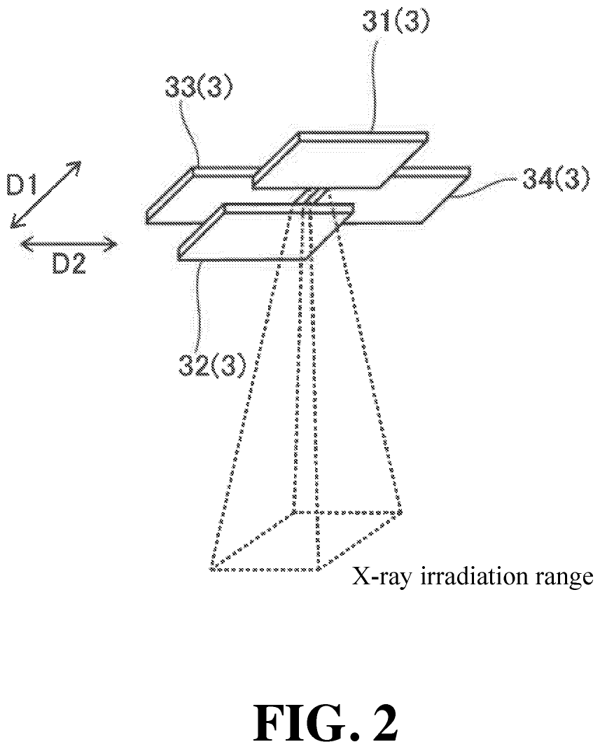

First, the configuration of an X-ray fluoroscope 100 according to this embodiment will be described with reference to FIG. 1 and FIG. 2.

As shown in FIG. 1, the X-ray fluoroscope 100 is configured to perform fluoroscopic inspection or image capturing of the imaging region (fluoroscopic region or image capturing region) of the subject by emitting X-rays toward the subject S. Further, the X-ray fluoroscope 100 is provided with an X-ray irradiation unit 1 configured to emit X-rays toward the subject S, an X-ray image receiving unit 2 configured to receive X-rays transmitted through the subject S, an MLC (Multi Leaf Collimator) 3 configured to narrow the irradiation range of X-rays irradiated from the X-ray irradiation unit 1, and a control unit 4 configured to control the opening degree of the MLC 3 by acquiring the X-ray fluoroscopic image I based on the X-ray received image of the X-ray image receiving unit 2 and control the irradiation intensity of X-rays irradiated from X-ray irradiation unit 1 based on the ROI (Region Of Interest) on the X-ray fluoroscopic image I. Note that the MLC 3 is an example of the “irradiation range control member” recited in claims. Further note that the ROI is an example of the “region of interest” recited in claims.

A display unit 5 for displaying the X-ray fluoroscopic image I and an operation unit 6 for operating the X-ray fluoroscope 100 are connected to the control unit 4. Further, the X-ray irradiation unit 1 and the X-ray image receiving unit 2 are arranged so as to be opposed by a C-arm 8. That is, this X-ray fluoroscope 100 shows an example of a so-called C-arm type X-ray fluoroscope. Further, a top board 7 for placing a subject S thereon is provided between the X-ray irradiation unit 1 and the X-ray image receiving unit 2 (between the MLC 3 and the X-ray image receiving unit 2).

The X-ray irradiation unit 1 includes an X-ray tube, which is not illustrated. The X-ray tube is configured to emit X-rays when thermal electrons jumped out of a negative electrode collide with a positive electrode by supplying a current to each of the internal positive electrode and the internal negative electrode to heat them and applying a voltage between the positive electrode and the negative electrode. In addition, X-rays generated in the X-ray tube are configured to be emitted toward the X-ray image receiving unit 2. Note that by changing the tube voltage applied to the X-ray tube, the irradiation intensity of X-rays to be irradiated is determined according to the tube voltage.

The X-ray image receiving unit 2 is configured by, for example, an FPD (Flat Panel Detector). Further, the FPD is configured to receive X-rays irradiated by the X-ray irradiation unit 1 and transmitted through the subject S and convert the received X-rays into electric signals. The FPD has an imaging element having a plurality of pixels (sections) inside, and is configured to detect the intensity of X-rays for each corresponding pixel and convert the X-ray information (detection signal) for each pixel into an electric signal (digital data) as a pixel value. The information of the X-rays converted into the electric signals is transmitted to the control unit 4.

As shown in FIG. 2, the MLC 3 includes four shielding plates 31, 32, 33, and 34 each made of a material (e.g., copper, lead, etc.) having a high X-ray shielding rate therein. The shielding plate 31 and the shielding plate 32 are each movable along the direction D1, and are arranged so as to face each other. Further, the shielding plate 33 and the shielding plate 34 are each movable along the direction D2 approximately orthogonal to the direction D1, and are arranged so as to face each other. The shielding plates 31 to 34 shield a part of the X-rays irradiated from the X-ray irradiation unit 1 to narrow the X-ray irradiation range (the range indicated by the dotted line in the figure). Further note that the direction D1 and the direction D2 are directions parallel to a plane (a plane substantially horizontal to the top board 7) substantially orthogonal to the X-ray irradiation direction.

The control unit 4 includes an X-ray irradiation unit driver 41 for driving the X-ray irradiation unit 1, an X-ray image receiving unit driver 42 for driving the X-ray image receiving unit 2, an MLC driver 43 for controlling the opening degree of the MLC 3, and a top board driver 47 for driving the top board 7. Note that the control unit 4 also includes an information processing apparatus such as a PC (personal computer). Specifically, the control unit 4 includes a storage unit 44 such as an HDD (hard disk drive) and a memory, an image processing unit 45, and a main control unit 46 such as a CPU (central processing unit). The image processing unit 45 may be an arithmetic processing unit dedicated to image processing, or may be made to function as an image processing unit 45 by making the CPU execute an image processing program. Alternatively, a device dedicated to image processing may be provided as the image processing unit 45.

The X-ray irradiation unit driver 41, the X-ray image receiving unit driver 42, the MLC driver 43, and the top board driver 47 are each configured by a potentiometer (not illustrated) or an encoder (not illustrated), a servomotor (not illustrated), and a moving mechanism (not illustrated) driven by the servomotor. The main control unit 46 performs rotational position control and rotational position acquisition of the servomotor based on the detection signal of the potentiometer or the encoder. As a result, the positions, velocities, and acceleration of the X-ray irradiation unit 1, the X-ray image receiving unit 2, and the top board 7 are controlled and acquired by the control unit 4. Further, the opening degree of the MLC 3 (shielding plates 31 to 34) is controlled and acquired by the control unit 4.

The positions of the X-ray irradiation unit 1, the X-ray image receiving unit 2, and the top board 7 are controlled to move in accordance with to the operation input (movement command) of the operator using the operation unit 6. Specifically, the X-ray irradiation unit 1 and the X-ray image receiving unit 2 which are connected to the C-arm 8 are configured so as to enable the translational movement in the X and Y directions and the rotational movement in the R direction about the longitudinal direction central axis of the top board 7 as a rotation axis. These movements are controlled by the X-ray irradiation unit driver 41 and the X-ray image receiving unit driver 42. The top board 7 is configured to enable the translational movement in the X, Y, and Z directions in FIG. 1. The movement of the top board 7 is controlled by the top board driver 47. Note that the top board 7 is configured to be manually moved by the operator.

Further, the opening degree of the MLC 3 controlled by the MLC unit driver 43 is set in accordance with the imaging region selected by the operator. Further, the opening degree of the MLC 3 is configured to be able to be changeable, at the beginning of or during the treatment/diagnosis, by an adjustment automatically performed by the control unit 4 based on the detection amount of X-rays transmitted through the imaging region of the subject S which changes by the range of the imaging region of the subject S or the thickness of the imaging region of the subject S reflected in the X-ray fluoroscopic image. Further, it is configured such that the opening degree can also be changed by the input of the operator.

The storage unit 44 stores various programs 441 executed by the main control unit 46 and the image processing unit 45, and various data 442 including data of the captured X-ray fluoroscopic image I and data of the preset ROI prepared in advance for each imaging region. The preset ROI is an example of the “initially set region of interest” recited in claims.

The image processing unit 45 sends out data or the like of the pixel value of the image in the set ROI to the storage unit 44 and the main control unit 46 based on the data of the X-ray fluoroscopic image I sent from the X-ray image receiving unit 2. The image processing unit 45 applies image processing to the X-ray fluoroscopic image I and makes the display unit 5 display the X-ray fluoroscopic image I.

The main control unit 46 makes the PC function as the control unit 4 of the X-ray fluoroscope 100 by executing the control program stored in the storage unit 44. The main control unit 46 controls the translational position or the rotational position of the X-ray irradiation unit driver 41, the X-ray image receiving unit driver 42, and the top board driver 47, and controls the opening degree of the MLC driver 43. Specifically, based on the detection signal of the encoder or the potentiometer provided on the X-ray irradiation unit driver 41, the X-ray image receiving unit driver 42, the MLC driver 43, and the top board driver 47, the main control unit 46 performs the drive control of the servomotor provided on each of them. The main control unit 46 also performs the control regarding the X-ray irradiation by the X-ray irradiation unit 1 and the readout control of the detection signal by the X-ray image receiving unit 2. Also, the main control unit 46 makes the X-ray irradiation unit 1 change the irradiation intensity of X-rays based on the data of the pixel value of the ROI on the X-ray fluoroscopic image transmitted from the image processing unit 45.

The display unit 5 is composed of an image display device such as a combination of a liquid-crystal monitor and a backlight, and performs a screen display based on the image output of the control unit 4. For example, the display unit 5 displays the captured X-ray fluoroscopic image I and performs various screen displays regarding the operation of the X-ray fluoroscope 100.

The operation unit 6 is composed of a keyboard, a mouse, an operation lever and the like for receiving the operation input by the user. The control unit 4 is configured, via the operation unit 6, to accept the mode selection of the imaging mode (which will be described later), the inputs of various imaging conditions, and the instruction for starting imaging and also accept the movement command of the X-ray irradiation unit 1, the X-ray image receiving unit 2, and the top board 7 and the registration of the selection of the imaging mode and the imaging region. When selecting a long imaging mode, which will be described later, as an imaging mode, an imaging range (a start point and an end point) may also be set.

The X-ray fluoroscope 100 is configured to image the subject S (subject to be imaged) lying on the top board 7. Specifically, the X-ray fluoroscope 100 is configured to capture the X-ray fluoroscopic image I by receiving X-rays irradiated from the X-ray irradiation unit 1 arranged above the top board 7 and transmitted through the subject S by the X-ray image receiving unit 2.

Further, the X-ray fluoroscope 100 is configured so as to be able to perform X-ray imaging (X-ray fluoroscopic imaging or X-ray image capturing) by arranging the X-ray irradiation unit 1 and the X-ray image receiving unit 2 at a predetermined position with respect to the subject S. In this case, the X-ray fluoroscope 100 can select, as an imaging mode, a fluoroscopic mode capable of acquiring an X-ray fluoroscopic image I in real time by sequentially capturing images at relatively low X-ray intensity and short time intervals.

Further note that the X-ray fluoroscope 100 can also select, as an imaging mode, a normal imaging mode for capturing an X-ray image by imaging a predetermined number of times at relatively high X-ray intensity. Furthermore, the X-ray fluoroscope 100 can select, as an imaging mode, a long imaging mode for capturing a long image with the body axis direction (X direction) of the subject S as the longitudinal direction by capturing a plurality of X-ray images sequentially (in a sequential order) while moving the X-ray irradiation unit 1 and the X-ray image receiving unit 2 relative to the subject S and combining the plurality of X-ray images.

The X-ray fluoroscope 100 is configured so that selection of the first control or the second control, the optimization of X-ray intensity, imaging time, imaging intervals, imaging timing, etc., can be set by the setting by the operator or the automatic control by the control unit in accordance with the imaging region of the subject S input by the operator. In this embodiment, the case is described in which X-ray fluoroscopy (imaging in the fluoroscopic mode) is performed to obtain an X-ray fluoroscopic image in real time in a state in which the X-ray irradiation unit 1, the X-ray image receiving unit 2, and the top board 7 are fixed at predetermined positions.

Here, the X-ray fluoroscope 100 according to this embodiment is configured to acquire the portion where the MLC 3 is reflected in the ROI on the X-ray fluoroscopic image I acquired from the X-ray image receiving unit 2 based on the opening degree of the MLC 3. Specifically, the control unit 4 (main control unit 46) is configured to acquire the positions of the shielding plates 31 to 34 based on the driving of the MLC driver 43. The driving of the MLC driver 43 is detected based on, for example, a potentiometer or an encoder equipped in the MLC driver 43. Specifically, it is configured to acquire the opening degree of the MLC 3 by acquiring the rotation angle of the servomotor equipped in the MLC driver 43 from the comparison of the response (response of the potentiometer or the encoder) to the voltage pulse applied at the initial position (e.g., fully closed state) and the movement position of the shielding plate 31 to 34.

Further, the control unit 4 (main control unit 46) is configured to set the range of the ROI. As a result, the control unit 4 is configured to acquire (calculate) the portion where the MLC 3 is reflected in the ROI on the X-ray fluoroscopic image I acquired from the X-ray image receiving unit 2 based on the acquired opening degree of the MLC 3 and the range of the set ROI. In the same manner as in the MLC driver 43, the X-ray irradiation unit driver 41, the X-ray image receiving unit driver 42, and the top board driver 47 are each configured to send the information on the translational position or the rotational position based on the response of the potentiometer or the encoder equipped in the respective drivers to the control unit 46.

In the X-ray fluoroscope 100 according to this embodiment, the control unit 4 is configured to be able to switch between the first control and the second control in accordance with the imaging region of the subject S. Specifically, at the start of treatment/diagnosis of the subject S, setting of the preset ROI optimized in advance and selection of the first control or the second control are performed in accordance with the selection of the imaging region. Hereinafter, the details of the first control and the second control will be described.

### (First Control)

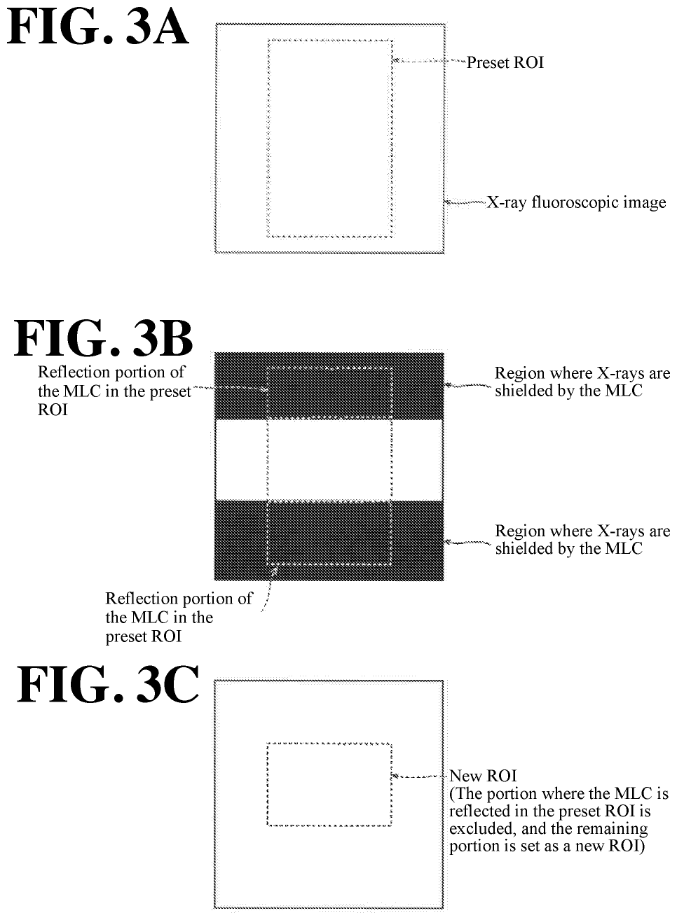

With reference to FIG. 3 to FIG. 5, the first control by the control unit 4 will be described. The first control is effective, for example, when the ROI of the MLC 3 is narrowed relatively small. For example, in the case of performing the treatment/diagnosis in a state in which the head is fixed, or in the case of performing a fluoroscopic inspection of the imaging region where the position is not required to be moved so much, the ROI is sometimes narrowed.

Here, in the X-ray fluoroscope 100 according to this embodiment, in cases where the MLC 3 is reflected in the ROI, the control unit 4 is configured to perform the first control to update the ROI to a new ROI by excluding the portion where the MLC 3 is reflected in the ROI.

Further, in the X-ray fluoroscope 100 according to this embodiment, in the case of performing the first control, when the MLC 3 is opened, the control unit 4 is configured to perform the control to update the region of interest to a new region of interest by returning the portion which was excluded from the updated region of interest among the portion where the MLC 3 is no longer reflected to the ROI again.

In the X-ray fluoroscope 100 according to this embodiment, the control unit 4 is configured to be able to switch between the first control and the second control in accordance with the imaging region of the subject S. The control unit 4 receives the selection of the imaging region of the subject S by the operator via the operation unit 6, and performs the range setting of the preset ROI and the selection of the first control or the second control in accordance with the imaging region of the subject S. Hereinafter, the case in which the first control is selected will be specifically described.

First, with reference to FIG. 3, when the MLC 3 is reflected in the preset ROI, the control to exclude the reflection portion of the MLC 3 from the set ROI will be described. As shown in FIG. 3A, at the start of using the X-ray fluoroscope 100, the control unit 4 acquires a preset ROI. On the X-ray fluoroscopic image I, the preset ROI is set as an ROI. At this time, the preset ROI and the set ROI match with each other.

Next, as shown in FIG. 3B, it is assumed that the opening degree of the MLC 3 is changed, so that reflection of the MLC 3 in the present ROI is caused. Note that the same control as described below is performed also in cases where the MLC 3 is reflected in the preset ROI at the time of acquiring the preset ROI. Further note that, as shown in FIG. 3B, in the region where the MLC 3 is reflected in the X-ray fluoroscopic image I, most of the X-rays to be irradiated are shielded, so almost no X-rays are detected. When the control unit 4 acquires the reflection of the MLC 3 (one or more of the shielding plates 31 to 34) in the preset ROI, the control unit 4 controls to set the portion where reflected portions of the MLC 3 are excluded from the preset ROI as a new ROI.

As a result, as shown in FIG. 3C, the ROI is updated to a new ROI where the regions where the ROI and the MLC 3 are common are removed. The control unit 4 is configured to repeat the control to acquire the position of the MLC 3 and update the ROI even after the update to a new ROI. Note that the opening degree of the MLC 3 (positions of the shielding plates 31 to 34) is acquired by the potentiometer or the encoder equipped in the MLC driver 43 as described above.

Next, a case will be described in which, after a new ROI is set, the opening degree of the MLC 3 is further changed, so the portion where the MLC 3 is reflected in the preset ROI is increased. Also in this case, the portion where the MLC 3 is reflected in the preset ROI is excluded and the control to update to a new ROI is further repeated, so the portion where the MLC 3 becomes newly reflected is also excluded from the ROI.

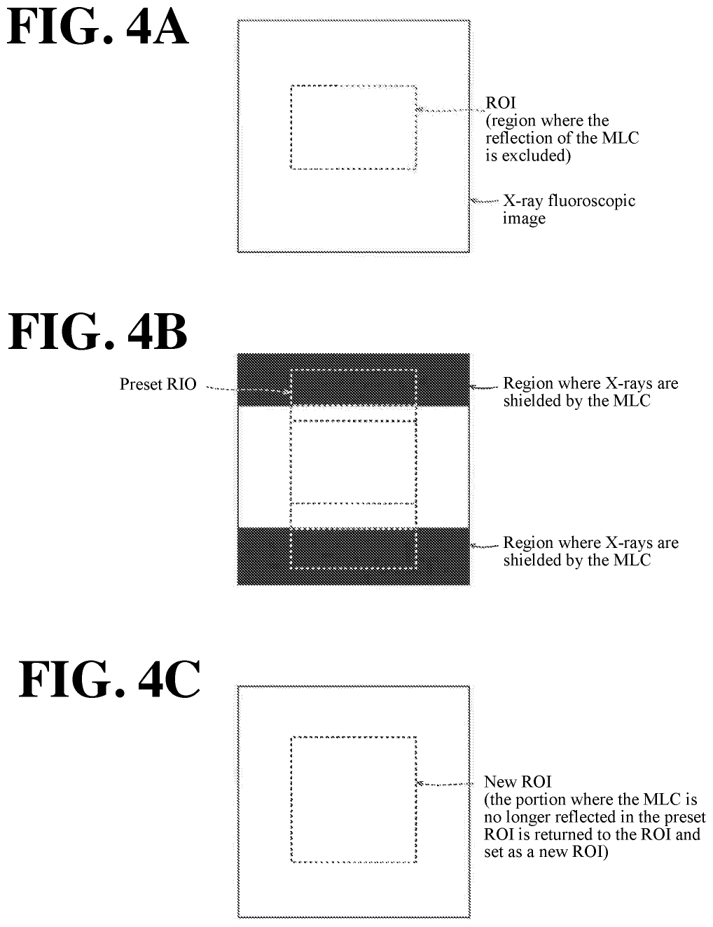

Next, with reference to FIG. 4, the control to recapture the portion where the MLC 3 disappears from the preset ROI when the portion where the MLC is reflected in the preset ROI is decreased after setting a new ROI will be described. As shown in FIG. 4A, on the X-ray fluoroscopic image I, an ROI is set in which reflected portions of the MLC 3 in the preset ROI are excluded. Here, as shown in FIG. 4B, also in cases where the portion where the MLC 3 is reflected in the preset ROI is decreased, the control unit 4 performs the control to acquire the portion where the MLC is reflected in the preset ROI after the decrease and exclude the portion where the MLC 3 is reflected in the preset ROI from the preset ROI. That is, as shown in FIG. 4C, the portion where the MLC is no longer reflected in the ROI is set as an ROI again, and therefore, in the updated ROI, the portion where the MLC 3 is no longer reflected in the preset ROI is returned.

In summary, the control unit 4 is configured to continuously perform the control to update an ROI to a new ROI by acquiring the portion where the MLC 3 is reflected in the preset ROI and excluding the portion where the MLC 3 is reflected in the preset ROI. As a result, the control is continuously performed in which the portion where the MLC 3 is reflected in the ROI is excluded in accordance with the change in the opening degree of the MLC 3 and the portion where the MLC 3 is no longer reflected in the ROI is returned up to the range of the preset ROI as a maximum region.

Here, in the X-ray fluoroscope 100 according to this embodiment, the control unit 4 is configured to adjust the intensity of X-rays irradiated from the X-ray irradiation unit 1 based on the mean value or the maximum value of the pixel values in the updated ROI.

Specifically, the control unit 4 is configured to continuously compare the mean value or the maximum value of the pixel values in the ROI in which the reflected portion of the MLC 3 is excluded with a target pixel value. When the pixel value in the set ROI is “brighter” than the target pixel value, it is considered that the X-ray irradiation intensity is too strong (X-ray irradiation is excessive), so the control unit 4 makes the X-ray irradiation unit 1 decrease the X-ray irradiation intensity. When the pixel value in the set ROI is “darker” than the target pixel value, it is considered that the X-ray irradiation intensity is too weak (X-ray irradiation is insufficient), so the control unit 4 makes the X-ray irradiation unit 1 increase the X-ray irradiation intensity. When the pixel value in the set ROI matches the target pixel value, it is considered that the X-ray irradiation intensity is optimal, so the control unit 4 makes the X-ray irradiation unit 1 maintain the X-ray irradiation intensity.

In the above description, the expressions “bright” and “dark” of the pixel value are replaced when the relationship between the negative and the positive of the image displayed in the display unit 5 is reversed, so the expressions are only convenient expressions for explaining the irradiation intensity of X-rays. In this embodiment, an example will be described in which the case in which X-rays to be received are strong, it is displayed brightly, and the case in which X-rays to be received are weak, it is displayed darkly.

That is, the X-ray irradiation intensity is set based on the data of the image in the ROI in which the portion where the MLC 3 is reflected in the (preset) ROI is excluded. Therefore, in the X-ray fluoroscope 100 of the present invention, the irradiation intensity of X-rays can be appropriately controlled. That is, the intensity of the X-ray irradiation will never be increased excessively based on the X-ray fluoroscopic image I that has become “darker” due to the reflection of the MLC 3 in the ROI. As a result, it is possible to acquire an X-ray fluoroscopic image I with good visibility suitable for the imaging region of the subject S.

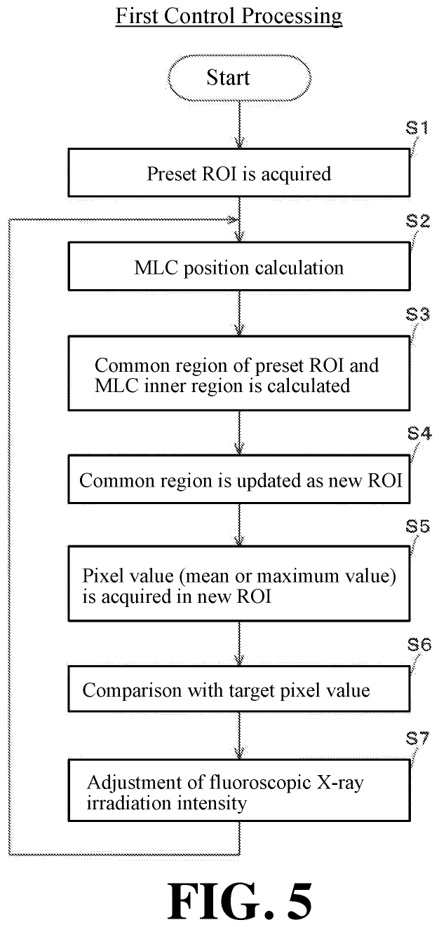

Hereinafter, with reference to FIG. 5, the flow of the first control is described using a flowchart. When the operator selects an imaging region at the start of use, a first control or a second control is started depending on the selected imaging region. Here, it is assumed that a first control is started. When the first control is started, in Step S1, the control unit 4 reads out the optimum preset ROI according to the imaging region from the storage unit 44, and the process proceeds to Step S2. At this time, the set ROI and the preset ROI match.

In Step S2, the control unit 4 acquires the position of the MLC 3 from the MLC driver 43 and calculates the position of the MLC, and the process proceeds to Step S3.

In Step S3, the control unit 4 calculates the common region between the preset ROI and the MLC inner region (the region in which the X-ray irradiation is not shielded by the MLC 3), and the process proceeds to Step S4.

In Step S4, the control unit 4 updates the set ROI to a new ROI which is the common region between the preset ROI and the MLC internal region, and the process proceeds to Step S5.

In Step S5, the control unit 4 acquires the pixel value in the updated new ROI, and the process proceeds to Step S6. Note that the pixel value acquired here is the mean value or the maximum value of the pixel value (for example, the luminance of the pixel) in the new ROI.

In Step S6, the control unit 4 compares the pixel value in the acquired new ROI with the preset target pixel value, and the process proceeds to Step S7. Note that the target pixel value is a predetermined pixel value adjusted in accordance with the imaging region so that the visibility of the X-ray fluoroscopic image I increases by bringing the pixel value in the ROI close to the target pixel value.

In Step S7, the control unit 4 makes the X-ray irradiation unit 1 maintain or change the X-ray irradiation intensity based on the comparison between the pixel value in the set ROI and the target pixel value, and the process returns to Step S2.

After that, Steps S2 to S7 of the first control are repeated until the fluoroscopic inspection by the X-ray fluoroscope 100 is completed. That is, since the opening degree of the MLC 3 is continuously calculated, the control to update the ROI to a new ROI in accordance with the change in the opening degree of the MLC 3 is continuously performed up to the range of the preset ROI as a maximum region. In addition, the X-ray irradiation intensity by the X-ray irradiation unit 1 is continuously changed as the opening degree of the MLC 3 or the pixel value in the ROI changes.

### (Second Control)

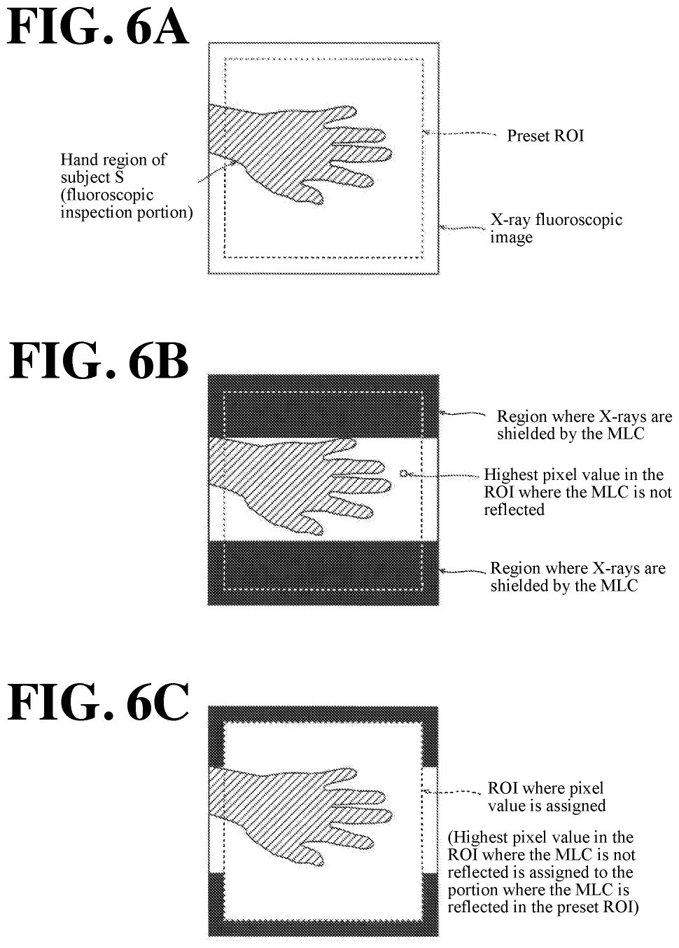

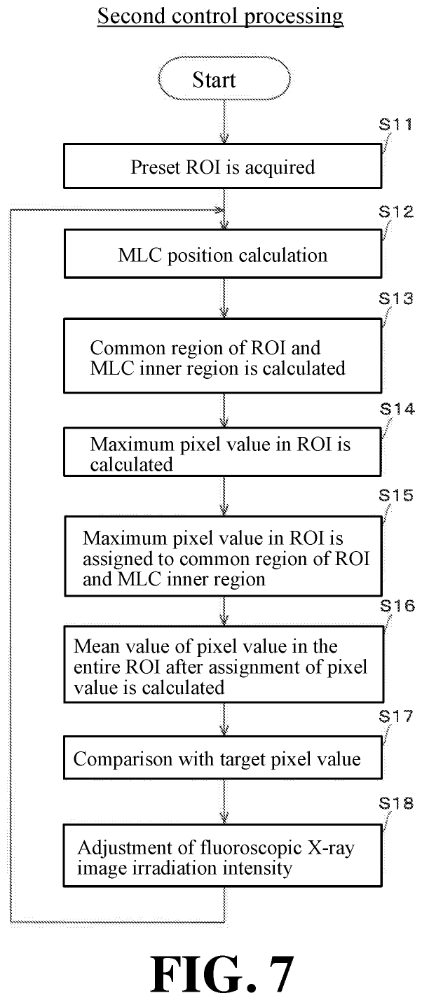

With reference to FIG. 6 and FIG. 7, a second control by the control unit 4 will be described. The second control is effective in cases where, for example, the ROI of the MLC 3 is taken relatively wide open. For example, in the case of the X-ray fluoroscopic inspection which requires to move the position of the treatment region of subject S greatly, such as, e.g., in the case of a surgery to fix bolts and/or wires to arms, legs, etc., the ROI is sometimes taken wide open.

Here, in the X-ray fluoroscope 100 according to this embodiment, the control unit 4 is configured to perform the second control in which a predetermined pixel value is assigned to the pixel of the portion where the MLC 3 is reflected in the ROI.

Further, in the X-ray fluoroscope 100 according to this embodiment, when performing the second control, the control unit 4 is configured to assign the maximum value of the pixel value of the portion where the MLC 3 is not reflected in the ROI to the portion where the MLC 3 is reflected in the ROI.

Hereinafter, the case in which the second control is selected will be specifically described. The description of contents common to the first control and the second control will be omitted as appropriate.

Descriptions will be made based on FIG. 6. As shown in FIG. 6A, at the start of using the X-ray fluoroscope 100, the control unit 4 acquires a preset ROI. Further, on the X-ray fluoroscopic image I, the preset ROI is set as an ROI. An imaging region (hand) of the subject S is reflected in the preset ROI. Note that in the second control, since the range of the set ROI is not changed, the set ROI and the preset ROI always match. Therefore, in the following description of the second control, in order to simplify the description, the preset ROI is simply described as the ROI.

As shown in FIG. 6B, the case in which the MLC 3 is reflected in the ROI will be considered. Note that, similar to the first control, also in cases where the MLC 3 is reflected in the ROI when the (preset) ROI is acquired, the same control will be performed. When the control unit 4 acquires the reflection of the MLC 3 (one or more of the shielding plates 31 to 34) in the ROI, the control unit 4 acquires the maximum value of the pixel value (for example, luminance, etc.) in the ROI. The control unit 4 assigns the acquired pixel value to the region where the MLC 3 is reflected in the ROI. Generally, it is considered that the pixel value of the portion where the MLC 3 is reflected is “darker” than the pixel value of the portion where the MLC 3 is not reflected. For this reason, it is considered that the maximum value of the pixel value of the ROI matches the maximum value of the pixel value of the portion where the MLC 3 is not reflected in the ROI as it is. Therefore, in this embodiment, the maximum value of the pixel value of the ROI is assigned as the maximum value of the pixel value of the portion where the MLC 3 is not reflected in the ROI. As a result, it is only necessary to acquire the maximum value of the pixel value of the ROI as a value to be assigned, so the processing amount in the control unit 4 can be reduced.

As a result, as shown in FIG. 6C, an image in which the above-described pixel value is assigned to the common region of the ROI and the reflection portion of the MLC 3 is obtained. Further note that the control unit 4 is configured to repeat the control to acquire the position of the MLC 3 and assign the pixel value to the ROI even after assigning the pixel value to the ROI.

The portion where the X-ray irradiation is shielded by the MLC 3 is considered to be a portion where the imaging region of the subject S almost does not exist (is not reflected). Therefore, for example, as shown in FIG. 6B and FIG. 6C, it is conceivable that the pixel values of most portions where the MLC 3 is reflected in the ROI can be approximately replaced with the pixel value of the brightest portion among the portions where the imaging region does not exist and the MLC 3 is not reflected in the ROI. Therefore, when considering luminance as a pixel value, the maximum value of the pixel value in the ROI is the value of the pixel (the “brightest” pixel) of the portion where X-rays are most strongly incident on the region where the MLC 3 is not reflected in the ROI. Note that it does not matter that the imaging region of the subject S is present in the region where the MLC 3 is reflected in the ROI.

In FIG. 6C, for the purpose of explanation, it is illustrated such that assignment of a pixel value is not performed at a portion where the MLC 3 is reflected in the X-ray fluoroscopic image I outside the ROI. However, it may be configured to perform the assignment of the pixel value also in a portion where the MLC 3 is reflected in the X-ray fluoroscopic image I outside the ROI. Further, the X-ray fluoroscopic image I to be displayed on the display unit 5 may be an X-ray fluoroscopic image before the assignment of the pixel value or may be an X-ray fluoroscopic image after the assignment of the pixel value.

Here, in the X-ray fluoroscope 100 of this embodiment, when the second control is selected, the control unit 4 is configured to adjust the intensity of the X-rays irradiated from the X-ray irradiation unit 1 based on the mean value or the maximum value of the pixel values of the entire ROI to which a predetermined pixel value is assigned.

Specifically, the control unit 4 is configured to continuously assign the maximum value of the pixel value of the portion where the MLC 3 is not reflected in the ROI as a predetermined value to the portion where the MLC 3 is reflected in the ROI and compare the mean value of the pixel values of the entire assigned ROI with the target pixel value. Moreover, the control unit 4 continuously acquires excess and deficiency of the X-ray irradiation intensity by the comparison with a target pixel value and control the X-ray irradiation intensity. That is, even in cases where there is a change in the opening degree of the MLC 3 or a variation in the pixel value of the portion where the MLC 3 is not reflected, the control unit 4 is configured to control to adjust the X-ray irradiation intensity appropriately in accordance with the change or variation.

As described above, the X-ray irradiation intensity is set based on the data of the image in which the maximum value of the pixel values of the ROI is assigned to the portion where the MLC 3 is reflected in the (preset) ROI. Therefore, in the X-ray fluoroscope 100 of the present invention, the irradiation intensity of X-rays can be controlled appropriately. That is, the intensity of the X-ray irradiation will never be increased excessively based on the X-ray fluoroscopic image I that has become “darker” due to the reflection of the MLC 3 in the ROI. As a result, it is possible to acquire an X-ray fluoroscopic image I with good visibility suitable for the imaging region of the subject S.

In the above-described configuration in which the maximum value of the pixel value of the portion where the MLC 3 is reflected in the ROI is assigned as a predetermined value, if the x-ray irradiation intensity is adjusted based on the maximum value of the pixel value of the entire ROI after assignment, it has the same effect as the case in which the irradiation intensity of the X-rays is adjusted based on the maximum value of the pixel value of the entire (initial) ROI before assignment. Therefore, the amount of processing unnecessarily increases. For this reason, in the case of adopting the configuration that the irradiation intensity of the X-rays is adjusted based on the maximum value of the pixel values of the entire ROI after assignment, the assignment of the pixel value may be omitted. As for the comparison of the mean value or the maximum value of pixel values in the ROI in which a predetermined value (the maximum value of pixel values in the portion where the MLC 3 is not reflected) is assigned to the reflected portion of the MLC 3 with the target pixel value, and the control of the X-ray irradiation intensity change, they are common to those in the first control and therefore the explanation will be omitted.

Hereinafter, with reference to FIG. 7, the flow of the second control is described using a flowchart. When the operator selects an imaging region at the start of use, the first control or the second control is started depending on the selected imaging region. Here, it is assumed that the second control is started. When the second control is started, in Step S11, the control unit 4 reads out the optimum (preset) ROI according to the imaging region from the storage unit 44, and the process proceeds to Step S12.

In Step S12, the control unit 4 acquires the position of the MLC 3 from the MLC driver 43, calculates the position of the MLC, and the process proceeds to Step S13.

In Step S13, the control unit 4 calculates the common region between the ROI and the MLC inner region (the region in which the X-ray irradiation is not shielded by the MLC 3), and the process proceeds to Step S14.

In Step S14, the control unit 4 acquires the maximum value of pixel values (for example, luminance, etc.) in the set ROI, and the process proceeds to Step S15.

In Step S15, the control unit 4 assigns the maximum value of pixel values in the acquired ROI to the common region between the preset ROI and the MLC inner region, and the process proceeds to Step S16.

In Step S16, the control unit 4 calculates the mean value of the pixel values of the pixels included in the entire ROI to which the pixel value is assigned, and the process proceeds to Step S17.

In Step S17, the mean value of the pixel values of the entire ROI to which the above calculated pixel value is assigned is compared with the preset target pixel value, and the process proceeds to Step S18.

In Step S18, the control unit 4 makes the X-ray irradiation unit 1 maintain or change the X-ray irradiation intensity based on the comparison between the pixel value in the set ROI and the target pixel value, and the process returns to Step S12.

After that, Steps S12 to S18 of the second control are repeated until the fluoroscopic inspection by the X-ray fluoroscope 100 is completed. That is, since the position of the MLC 3 is continuously calculated, the control is continuously performed in which the maximum value of the pixel values in the portion where the MLC 3 is not reflected in the ROI is assigned to the pixel value of the portion where the MLC 3 is reflected in the ROI as the opening degree of the MLC 3 changes. Further, the X-ray irradiation intensity by the X-ray irradiation unit 1 is continuously changed as the opening degree of the MLC 3 changes or the pixel value in the ROI changes.

The description of contents common to the first control and the second control will be omitted as appropriate.

### (Effects of Embodiment)

In the embodiment according to the present invention, the following effects can be obtained.

In the embodiment according to the present invention, as described above, the control unit 4 is provided to the X-ray fluoroscope 100. The control unit 4 controls the opening degree of the MLC 3 by acquiring the X-ray fluoroscopic image I based on the X-ray received image of the X-ray image receiving unit 2 and control the irradiation intensity of X-rays irradiated from the X-ray irradiation unit 1 based on the ROI on the X-ray fluoroscopic image I. Further, the control unit 4 is configured, when the MLC 3 is reflected in the ROI set on the X-ray fluoroscopic image I, to perform at least one of the first control that excludes the portion where the MLC 3 is reflected in the ROI and updates to a new ROI and the second control that assigns a predetermined pixel value to the pixel of the portion where the MLC 3 is reflected in the region of interest.

With this, when performing the first control, since the portion where the MLC 3 is reflected in the ROI is excluded, the excess and deficiency of the X-ray irradiation intensity can be acquired appropriately based on only the portion where the irradiation range control member is not reflected in the ROI.

Further, when performing the second control, since the pixel value when there is no reflection of the MLC 3 is assigned to the portion where X-rays are not detected by the reflection of the MLC 3, the excess and deficiency of the X-ray irradiation intensity can be appropriately acquired based on the ROI. As a result, even when there occurs a portion where the X-ray image receiving unit 2 hardly detects X-rays due to the reflection of the MLC 3 in the ROI, it is possible to suppress that the intensity of the X-rays irradiated to perform the fluoroscopic inspection of the subject S is excessively increased. With this, even when the MLC 3 (irradiation range control member) is reflected in the region of interest, it is possible to suppress deterioration of the visibility of the X-ray fluoroscopic image I. Further, since the X-ray irradiation intensity can be suppressed from being excessively increased, the exposure dose of the operator of the X-ray fluoroscope 100 and the patient (subject S) can be reduced.

Further, the control unit 4 is configured to acquire the portion where the MLC 3 is reflected in the ROI on the X-ray fluoroscopic image I acquired from the X-ray image receiving unit based on the opening degree of the MLC 3. With this, it is possible to easily acquire the portion where the MLC 3 is reflected in the ROI from the opening degree of the MLC 3. Further, since the opening degree of the MLC 3 is controlled by the control unit 4, the control unit 4 can easily acquire the opening degree of the MLC 3.

Further, the control unit 4 is configured to adjust the intensity of the X-rays irradiated from the X-ray irradiation unit 1 based on the mean value or the maximum value of the pixel values of the entire region of interest to which the pixel value in the updated ROI or the predetermined pixel value is assigned. With this, in the case of controlling based on the mean value of the pixel values in the updated ROI or the pixel values of the entire ROI to which the predetermined pixel value is assigned, the X-ray intensity can be adjusted based on the pixel value of the entire ROI. Further, in the case of controlling based on the maximum value of the pixel values in the updated ROI or the pixel values of the entire ROI to which the predetermined pixel value is assigned, for example, when considering the luminance as the pixel value, the X-ray intensity can be adjusted based on the brightest pixel value in the ROI.

Further, when performing the first control, the control unit 4 is configured to perform the control to update the portion which was excluded from the updated ROI among the portion where the MLC 3 is no longer reflected to a new ROI by returning it to the RIO up to the preset ROI as a maximum region. With this, even in cases where the MLC 3 material once closed during the fluoroscopic inspection of the subject S is opened again, the excluded portion is returned to the ROI. Therefore, the control unit 4 can update (expanded) the ROI following the spread of the irradiation range. Also, since the ROI is updated (expanded) up to the preset ROI as a maximum region, it is possible to prevent the ROI from being expanded excessively beyond an appropriate range.

Further, in the case of performing the second control, the control unit 4 is configured to assign the maximum value of the pixel values of the portion where the MLC 3 is not reflected in the ROI to the portion where the MLC 3 is reflected in the ROI. With this, the maximum value of the portion where the MLC 3 is not reflected in the ROI is assigned to the portion where the MLC 3 is reflected in the ROI, it is possible to easily assign the hypothetical pixel value of the entire ROI when the MLC 3 is not reflected to the portion where the MLC 3 is reflected in the ROI.

Specifically, it is considered that there exists no subject S to be fluoroscopically inspected in most of the portion where irradiation is shielded by the MLC 3, so the maximum value of the pixel values of the portion where the MLC 3 is not detected, which is assumed to be the portion where X-rays are hardly transmitted through the subject S and received by a receiver, is assigned to the portion where the MLC 3 is reflected in the ROI. With this, since it is possible to switch between the first control and the second control according to the imaging region of the subject S, a more desirable X-ray fluoroscopic image can be acquired according to the situation and the purpose of the portion to be fluoroscopically inspected.

### (Modifications)

It should be understood that the embodiments disclosed here are examples in all respects and are not restrictive. The scope of the present invention is shown by the scope of the claims rather than the descriptions of the embodiments described above, and includes all changes (modifications) within the meaning of equivalent and the scope of claims.

For example, in the above embodiment, an example is shown in which the X-ray fluoroscope 100 performs both the first control and the second control, but the present invention is not limited to this. In the present invention, the X-ray fluoroscope 100 may be configured to perform only either one of the first control and the second control.

Also, in the above embodiment, an example is shown in which when performing the first control, the X-ray irradiation intensity is controlled based on the mean value or the maximum value of the pixel values of the ROI updated in the common area between the preset ROI and the portion where the MLC 3 is reflected, but the present invention is not limited to this. In the present invention, the X-ray irradiation intensity may be controlled based on the pixel value calculated other than the calculation of taking the average or the maximum of the pixel values in the updated ROI in the common region of the preset ROI and the portion where the MLC 3 is reflected.

In the above embodiment, although an example is shown in which the maximum value of the pixel values in the entire ROI is assigned as a predetermined value to be assigned to the portion where the MLC 3 is reflected in the ROI when performing the second control, the present invention is not limited to this.

In the present invention, it may be configured such that the maximum value of the pixel values only in the portion where the MLC is not reflected in the ROI is assigned as a predetermined value to be assigned to the portion where the MLC 3 is reflected in the ROI.

Further, when performing the second control, it may be configured such that for example, a constant value (pixel value) adjusted in advance for each imaging region may be assigned as a predetermined value to be assigned to the portion where the MLC 3 is reflected in the ROI or it may be configured such that the pixel value acquired by performing calculation other than taking the maximum value is assigned to the pixel value of the portion where the MLC 3 is not reflected in the ROI.

Further, in the above embodiment, an example is shown in which when performing the second control, the X-ray irradiation intensity is controlled based on the mean value of the pixel values in the ROI to which a predetermined value is assigned to the common region of the ROI and the portion where the MLC 3 is reflected, but the present invention is not limited to this. In the present invention, the X-ray irradiation intensity may be controlled based on the pixel value calculated other than the calculation of taking the mean value of the pixel values in which a predetermined value is assigned to the common region of the ROI and the portion where the MLC 3 is reflected.

Moreover, in the aforementioned embodiment, although an example is shown in which the MLC is provided with four pieces of shielding plates 31, 32, 33, and 34, the present invention is not limited to this. The MLC 3 of the present invention may be provided with four or more shielding plates. In this case, a direction D3 different from the directions D1 and D2 in the figure may be newly set, and a shielding plate may be provided along the direction D3. Further, the shielding plates 31 to 34 may be configured such that each plate is divided into a plurality of sheets in the direction orthogonal to the movement direction in the horizontal plane, and each divided shielding plate can be moved independently. In cases where the shielding plate is divided as described above, it is usually sufficient to provide the shielding plate in one direction (for example, the direction D1). In FIG. 2, the shielding plate is shown as a thin plate with respect to the X-ray irradiation direction, but it may be configured as a thick plate with respect to the X-ray irradiation direction.

In the above embodiment, an example is shown in which the control unit 4 (main control unit 46) is configured to acquire the opening degree of the MLC 3 based on the positions of shielding plates 31 to 34 acquired by the potentiometer or the encoder provided in the MLC driver 43, but the present invention is not limited to this. In the present invention, it may be configured such that the positions of the shielding plates 31 to 34 are acquired by another configuration in which, for example, the opening degree of the MLC 3 is acquired by an optical detector. Further, it may be configured such that a threshold value of a pixel value is set in the X-ray fluoroscopic image I and the portion having a pixel value lower than the threshold value is determined as a portion where the MLC 3 is reflected.

In the above embodiment, an example is shown in which the preset ROI is set automatically in accordance with the selection of the imaging region, but the present invention is not limited to this. In the present invention, the preset ROI may be set by, for example, the range specification by the operator, or may be set by being selected the operator among several preset ROIs.

In the above embodiment, an example is shown in which the head or the hand of the subject S is fluoroscopically inspected by the X-ray fluoroscope 100, but the present invention is not limited thereto. In the present invention, the X-ray fluoroscope 100 may be configured such that the imaging region (fluoroscopic inspection region), such as, e.g., the lumbar spine, the arm, the leg, the chest, and the abdomen of the subject S, can be selected. In addition to selecting an imaging region, an adjustment may be made based on the individual difference, such as, e.g., the body shape. Note that the X-ray fluoroscope 100 of the present invention may be used in an angiography device or the like for X-ray imaging blood vessels using a contrast agent.

Also note that, for the sake of explanation, for convenience, it has been described that the first control is performed when a head is fluoroscopically inspected and the second control is performed when a hand is fluoroscopically inspected, but it may be configured to perform the second control for fluoroscopically inspecting a head and perform the first control for fluoroscopically inspecting a hand. Further note that an example is directed to an embodiment in which the present invention is applied when performing the fluoroscopic mode as an imaging mode, but the present invention may be used for a normal imaging mode and a long imaging mode. That is, the present invention can be applied not only to the X-ray fluoroscopy but also to the X-ray imaging. In addition, the present invention may be applied to the X-ray fluoroscope 100 that performs only X-ray fluoroscopy (fluoroscopic imaging mode).

In the above embodiment, the C-arm type X-ray fluoroscope in which the X-ray irradiation unit 1 and the X-ray image receiving unit 2 are supported by the C-arm 8 is exemplified, but the present invention is not limited to this. In the present invention, for example, the present invention may be applied to an island-type X-ray fluoroscope. Further, although an example is shown in which the subject S (a person targeted for a medical treatment, a medical examination, etc.) is fluoroscopically inspected in a recumbent position, but it may be configured such that the subject S is fluoroscopically inspected in a sitting or standing position.

In the above embodiment, for convenience of explanation, although the first control and the second control by the control unit 4 are described using the “flow driven type” flowchart, the present invention is not limited to this. The processing of the control unit 4 may be performed by an “event-driven type” that executes on an event basis. In this case, it may be performed in a completely event driven manner, or a combination of event driving and flow driving may be performed.

1. An X-ray fluoroscope comprising:
an X-ray irradiation unit configured to irradiate X-rays to a subject;
an X-ray image receiving unit configured to receive X-rays transmitted through the subject;
an irradiation range control member configured to narrow an irradiation range of the X-rays irradiated from the X-ray irradiation unit; and
a control unit configured to control an opening degree of the irradiation range control member by acquiring an X-ray fluoroscopic image based on an X-ray image of the X-ray image receiving unit and control irradiation intensity of X-rays irradiated from the X-ray irradiation unit based on a region of interest on the X-ray fluoroscopic image,
wherein when the irradiation range control member is reflected in the region of interest, the control unit is configured to perform at least one of a first control to update the region of interest to a new region of interest by excluding a portion where the irradiation range control member is reflected in the region of interest and a second control to assign a predetermined pixel value to a pixel of the portion where the irradiation range control member is reflected in the region of interest.

2. The X-ray fluoroscope as recited in claim 1,
wherein the control unit is configured to acquire a portion where the radiation range control member is reflected in the region of interest on the X-ray fluoroscopic image acquired from the X-ray image receiving unit based on the opening degree of the irradiation range control member.

3. The X-ray fluoroscope as recited in claim 1,
wherein the control unit is configured to adjust the irradiation intensity of the X-rays irradiated from the X-ray irradiation unit based on a mean value or a maximum value of pixel values of the updated region of interest or pixel values of the entire region of interest to which a predetermined pixel value is assigned.

4. The X-ray fluoroscope as recited in claim 1,
wherein when the irradiation range control member is opened when performing the first control, the control unit is configured to control to update the region of interest to a new region of interest by returning a portion which was excluded from the updated region of interest among a portion where the irradiation range control unit is no longer reflected in the region of interest again to a new region of interest up to an initially set region of interest as a maximum region.

5. The X-ray fluoroscope as recited in claim 1,
wherein when performing the second control, the control unit is configured to assign a maximum value of the pixel value of a portion where the irradiation range control member is not reflected in the region of interest to the portion where the irradiation range control member is reflected in the region of interest.

6. The X-ray fluoroscope as recited in claim 1,
wherein the control unit is configured to be able to switch between the first control and the second control in accordance with an imaging region of the subject.
